(19) United States
(12) Patent Application Publication
(10) Pub. No.: US 2025/0161234 A1
(43) Pub. Date: May 22, 2025

(54) METHODS OF USE OF BENZALDEHYDE COMPOSITIONS

(71) Applicants: Shmuel ROBINOV, Petach Tikvah (IL); Alexander Saadia ROBINOV, Petach Tikvah (IL); Bronya ELIEZER, Hadera (IL); Chaim Avihai SIMNER, Ein Vered (IL)

(72) Inventors: Shmuel ROBINOV, Petach Tikvah (IL); Alexander Saadia ROBINOV, Petach Tikvah (IL); Bronya ELIEZER, Hadera (IL); Chaim Avihai SIMNER, Ein Vered (IL)

(21) Appl. No.: 18/841,460

(22) PCT Filed: Feb. 24, 2023
(86) PCT No.: PCT/IB2023/051706

§ 371 Date: Aug. 26, 2024

Related U.S. Application Data
(60) Provisional application No. 63/313,371, filed on Feb. 24, 2022.

(51) Int. Cl. A61K 31/11; A23B 2/75; A23B 4/20; A23B 45/70; A61K 36/738; A61K 45/06; A61P 31/14; B65D 81/28

(52) U.S. Cl. CPC A61K 31/11; A23B 2/75; A23B 4/20; A23B 45/70; A61K 36/738; A61K 45/06; A61P 31/14; B65D 81/28; A61K 2236/55

(57) ABSTRACT

Provided are compositions comprising benzaldehyde and a carrier for use in methods of treating a condition in a subject in need thereof by administration of the composition; methods for controlling growth of a plant by contacting the plant with the composition; methods for disinfection of a surface by contacting the surface with the composition; methods for preserving food by introducing the composition into the vicinity of the food; as well as kits and packages comprising the composition.

### CROSS-REFERENCE TO RELATED APPLICATION

The present application gains priority from U.S. Provisional Application No. 63/313,371 filed Feb. 24, 2022 which is incorporated by reference as if fully set-forth herein.

### FIELD OF THE INVENTION

The present invention, in at least some embodiments, relates to methods of use of benzaldehyde and more specifically to compositions comprising benzaldehyde and a carrier for use in methods of treating a condition in a subject, methods for controlling growth of a plant, methods for disinfection, methods for preserving food or preserving tissue; as well as to kits and packages comprising the composition.

### BACKGROUND OF THE INVENTION

Rosales is an order of flowering plants. It contains about 7,700 species, divided into about 260 genera. Rosales comprise nine families, including the rose family, Rosaceae. Well-known members of Rosales include: roses, strawberries, blackberries and raspberries, apples and pears, plums, peaches and apricots, almonds, rowan and hawthorn, jujube, elms, banyans, figs, mulberries, breadfruit, nettles, hops, and cannabis.

One of the substances that can be found in the fruit pit or seed of Rosales is Benzaldehyde (C₆H₅CHO), an aromatic aldehyde bearing a single formyl group and having a characteristic bitter almond odor. Benzaldehyde may be obtained from the plant in the form of a clear colorless to yellow liquid by isolation methods such as extraction or distillation.

### BRIEF DESCRIPTION OF THE DRAWINGS

Some embodiments of the invention are described herein with reference to the accompanying figures. The description, together with the figures, makes apparent to a person having ordinary skill in the art how some embodiments of the invention may be practiced. The figures are for the purpose of illustrative discussion and no attempt is made to show structural details of an embodiment in more detail than is necessary for a fundamental understanding of the invention. For the sake of clarity, some objects depicted in the figures are not to scale.

FIG. 1A presents microbiological test results obtained 10 minutes after treatment of killed fish with a composition comprising benzaldehyde in ice water, in accordance with an embodiment of the present invention;

FIG. 1B presents microbiological test results obtained after 5 days of treatment with a composition comprising benzaldehyde in ice water, in accordance with an embodiment of the present invention;

FIG. 1C presents microbiological test results obtained after 12 days of treatment with a composition comprising benzaldehyde in ice water, in accordance with an embodiment of the present invention; and

FIG. 2 presents photographic data showing the difference in preservation of a food product with and without treatment with a food preservative composition comprising benzaldehyde, in accordance with an embodiment of the present invention.

### SUMMARY OF THE INVENTION

According to an aspect of some embodiments of the present invention, there is provided a method of treating or preventing a condition in a subject in need thereof, the method comprising administering to the subject a composition comprising benzaldehyde and a carrier.

According to some embodiments, the condition is selected from the group consisting of cancer, heart disease, cataracts, poor eyesight, an immune deficiency, poor brain function, an inflammatory condition, a viral condition, a bacterial condition, hypercholesterolemia, poor iron absorption, a respiratory disease, arteriosclerosis, dementia, tissue damage, skin damage due to exposure to sun, diabetes, age-related degenerative disorders, stroke, cerebrovascular incidents, asthma and shortness of breath, and poor respiratory system.

According to some embodiments, the cancer is selected from the group consisting of breast cancer, ovarian cancer, lung cancer, uterine cancer, and lungs, helps prevent uterine cancer, breast cancer, prostate cancer, lung cancer and cancer of the large intestine.

According to some embodiments, the viral condition comprises coronavirus infection.

According to some embodiments, the composition is administered in a food.

According to some embodiments, benzaldehyde is present in the composition at a concentration of from about 0.08 wt % to about 50 wt %.

According to some embodiments, benzaldehyde is present in the composition at a concentration of about 10 wt %.

According to some embodiments, the composition is administered at a daily dosage of from about 0.5 ml to about 1000 ml.

According to some embodiments, the composition is provided in a form selected from the group consisting of a solid, a semi-solid, a liquid and a vapor.

According to some embodiments, the solid form comprises a powder.

According to some embodiments, the carrier is water and the composition comprises an emulsion.

According to some embodiments, the carrier comprises an organic solvent and the composition comprises a homogenous solution.

According to some embodiments, administering comprises oral administration.

According to some embodiments, the composition is a liquid and administering comprises inhalation of a vapor produced from said composition.

According to some embodiments, the semi-solid form is a cream and the method comprises topical administration.

According to some embodiments, the composition further comprises vitamin C.

According to an aspect of some embodiments of the present invention, there is provided a method of controlling growth of a plant, comprising contacting the plant with a composition comprising benzaldehyde and a carrier.

According to an aspect of some embodiments of the present invention, there is provided a method of preserving food comprising introducing a composition comprising benzaldehyde into the vicinity of the food.

According to an aspect of some embodiments of the present invention, there is provided a method of disinfecting a surface, comprising contacting the surface with a composition comprising benzaldehyde and a carrier.

According to some embodiments, the surface is selected from the group consisting of an area of skin of an animal and a surface of an inanimate object.

According to some embodiments, the benzaldehyde is synthetic benzaldehyde.

According to some embodiments, the benzaldehyde is isolated from a part of a plant of the order Rosales, wherein the part of the plant is selected from the group consisting of a pit, a seed and a fruit.

According to some embodiments, the benzaldehyde is isolated by a method selected from the group consisting of steam distillation and extraction.

According to an aspect of some embodiments of the present invention, there is provided a nutritional supplement comprising a composition comprising benzaldehyde and a carrier.

According to an aspect of some embodiments of the present invention, there is provided a kit for use in treating or preventing an infection in a subject in need thereof, the kit comprising benzaldehyde and a vaporizer, said vaporizer having a receptacle configured to receive therein a composition comprising the benzaldehyde and to vaporize said composition.

According to some embodiments, the benzaldehyde is provided as a composition in a liquid carrier.

According to some embodiments, the kit further comprise a liquid carrier for combination with the benzaldehyde in the receptacle.

According to an aspect of some embodiments of the present invention, there is provided a food package comprising an inner volume for containing therein a food product and a composition comprising benzaldehyde and a carrier.

According to some embodiments, the composition is separated from the inner volume by a removable barrier.

### DETAILED DESCRIPTION OF SOME EMBODIMENTS OF THE INVENTION

The invention, in some embodiments, relates to methods of use of a composition comprising benzaldehyde.

The invention provides novel uses of fruit, pit or seed extracts of Rosales, such as raw bitter almonds, apples, peaches etc. The fruit, pit or seed extract include a plurality of components including, for example, Benzaldehyde (C₆H₅CHO). The present Inventors have surprisingly identified that Benzaldehyde (C₆H₅CHO), optionally in the absence of any additional components of Rosales, is effective in treatment and prevention of a wide range of conditions and symptoms thereof in animals, including human animals, resulting in improved health of the animal.

The principles, uses and implementations of the teachings of the invention may be better understood with reference to the accompanying description and figures. Upon perusal of the description and figures present herein, one skilled in the art is able to implement the teachings of the invention without undue effort or experimentation. In the figures, like reference numerals refer to like parts throughout.

Before explaining at least one embodiment of the invention in detail, it is to be understood that the invention is not necessarily limited in its application to the details of construction and the arrangement of the components and/or methods set forth herein. The invention is capable of other embodiments or of being practiced or carried out in various ways. The phraseology and terminology employed herein are for descriptive purpose and should not be regarded as limiting.

The description is not to be taken in a limiting sense but is made merely for the purpose of illustrating the general principles of the invention, since the scope of the invention is best defined by the appended claims.

Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention.

According to some embodiments, the benzaldehyde used in the composition of the present invention comprises purified benzaldehyde. As used herein, the term “purified benzaldehyde” refers to benzaldehyde from which at least one additional component originally present in the plant is absent, and is intended to refer both to synthetic benzaldehyde, wherein the process of synthesis used results in the purified benzaldehyde and to purified benzaldehyde isolated from a plant, such as by distillation or extraction. According to some such embodiments, the benzaldehyde is provided at a purity of at least 75%, at least 80%, at least 85%, at least 90%, at least 95%, at least 99%, or at least 99.9%.

As used herein, the term “food” is intended to include both solid and liquid food (including beverages) for human consumption and animal feed. As used herein, the term “animal feed” refers to a product intended for consumption by a non-human animal, such as livestock, poultry, companion animal, or the like.

As used herein, the term “treating” includes ameliorating, mitigating, and reducing the instances of a disease or condition, or the symptoms of a disease or condition. In some embodiments, the treating refers to medical treatment i.e. treatment relating to the health of the subject. In some embodiments, the treating refers to cosmetic treatment i.e. treatment relating to aesthetic, non-medical aspects of a skin condition.

As used herein, the term “administering” includes any mode of administration, such as oral, subcutaneous, sublingual, transmucosal, parenteral, intravenous, intra-arterial, buccal, sublingual, topical, vaginal, rectal, ophthalmic, otic, nasal, inhaled, intramuscular, intraosseous, intrathecal, and transdermal, or combinations thereof. “Administering” can also include providing a different compound that when ingested or delivered as above will necessarily transform into the compound that is desired to be administered, this type of “different compound” is often being referred to as a “Prodrug”. “Administering” can also include prescribing or filling a prescription for a dosage form comprising a particular compound. “Administering” can also include providing directions to carry out a method involving a particular compound or a dosage form comprising the compound or compounds.

As used herein, the term “therapeutically effective amount” means the amount of an active substance that, when administered to a subject for treating a disease, disorder, or other undesirable medical condition, is sufficient to have a beneficial effect with respect to that disease, disorder, or condition. The therapeutically effective amount will vary depending on the chemical identity and formulation form of the active substance, the disease or condition and its severity, and the age, weight, and other relevant characteristics of the patient to be treated. Determining the therapeutically effective amount of a given active substance is within the ordinary skill of the art and typically requires no more than routine experimentation.

As used herein, the terms “comprising”, “including”, “having” and grammatical variants thereof are to be taken as specifying the stated features, integers, steps or components but do not preclude the addition of one or more additional features, integers, steps, components or groups thereof. These terms encompass the terms “consisting of” and “consisting essentially of”.

As used herein, the indefinite articles “a” and “an” mean “at least one” or “one or more” unless the context clearly dictates otherwise.

As used herein, when a numerical value is preceded by the term “about”, the term “about” is intended to indicate +/−10% of that value.

According to an aspect of some embodiments of the present invention, there is provided a method of treating or preventing a condition in a subject in need thereof, the method comprising administering to the subject a composition comprising benzaldehyde and a carrier.

According to an aspect of some embodiments of the present invention, there is provided a composition comprising benzaldehyde and a carrier, for use in therapy.

The present inventors have discovered novel advantages and/or uses for the composition comprising benzaldehyde (which may either be an extract of a phytochemical comprising benzaldehyde in which benzaldehyde is the primary component or synthetically produced pure benzaldehyde) and for the methods of using this composition, in its various concentrations and dosages described herein. Such uses include the following: affords protection against various types of cancer in the person (or other mammalian subject) including breast, ovary and lungs, helps prevent uterine cancer, breast cancer, prostate cancer, lung cancer, cancer of the large intestine, reduces the growth of tumors, prevents heart disease, helps reduce the development of cataracts, in general protects eyesight, has strong antioxidant activity, neutralizes the activity of free radicals, triggers the activity of the immune system, improves brain function, counteracts the aging process and fights inflammation (is anti-inflammatory), counteracts viruses and bacteria, regulates the production of cholesterol, prevents general heart disease, improves the body's iron absorption, prevents respiratory disease, influences the metabolism of cholesterol and estrogen so as to improve health, acts as a medication to lower the amount of the type of cholesterol that is not good for the body and/or to improve the ratio of good cholesterol to bad cholesterol within the human body, strengthens blood vessels, especially capillaries, protects against arteriosclerosis and in general against internal diseases connected to blood flow, is helpful against dementia, facilitates tissue rehabilitation especially collagen fiber and improves the function of the endothelium that is liable to be damaged by risk factors of arteriosclerosis, protects the skin from damage by the sun, promotes healing of tissue, slows down the aging process with respect to the skin and eye, prevents complications associated with diabetes, helps prevent age-related degenerative disorders, protects against stroke and cerebrovascular accidents, is helpful against asthma and shortness of breath, and strengthens the respiratory system.

Accordingly, in some embodiments, the composition comprising benzaldehyde as disclosed herein in the dosages described herein is for administration on a daily basis. In some embodiments, the composition is for administration twice a day, three times a day, four times a day, four times a week, three times a week, four times a week, weekly, biweekly, monthly or as required. Furthermore, the above advantages apply as well to animals and to plants.

According to some embodiments, the condition is selected from the group consisting of cancer, heart disease, cataracts, poor eyesight, an immune deficiency, poor brain function, an inflammatory condition, a viral condition, a bacterial condition, hypercholesterolemia, poor iron absorption, a respiratory disease, arteriosclerosis, dementia, tissue damage, skin damage due to exposure to sun, diabetes, age-related degenerative disorders, stroke, cerebrovascular incidents, asthma and shortness of breath, and poor respiratory system.

According to some embodiments, the cancer is selected from the group consisting of breast cancer, ovarian cancer, lung cancer, uterine cancer, and lungs, helps prevent uterine cancer, breast cancer, prostate cancer, lung cancer and cancer of the large intestine.

According to some embodiments, the viral condition comprises a coronavirus infection, such as COVID19 infection.

According to some embodiments, the composition as disclosed herein is used to maintain or improve the health of a subject, including by preventing development of a pathological condition.

According to some embodiments, the composition as disclosed herein is used as an antimicrobial agent, and the method of treating comprises preventing or treating a microbial infection, including a bacterial, viral or fungal infection.

According to some embodiments, the bacterial infection is an infection by bacteria selected from the group consisting of Listeria spp., Salmonella ISO. E. coli, Sulfite reducing clostridia, Staphylococcus aureus. Coliforms, Enterococcus feaclis, Pseudomonas aeruginosa, Klebsiella pneumoniae.

According to some embodiments, the composition is used in a method of strengthening the immune system. As used herein, the term “strengthening the immune system” is intended to refer to improving the functioning of the immune system of a subject, such as by increasing the innate T-cell-mediated immune response and/or the adaptive antibody response, resulting in improved resistance of the subject to infection by extracellular pathogens. According to some embodiments, the effect is achieved via increased production of cytokines and/or prostaglandins. According to some embodiments, the immune system is strengthened by preventing or reducing growth of microorganisms such as bacteria, fungi and viruses, such that replication of cells of the immune system is slowed down, resulting in improved function.

According to some embodiments, wherein the condition is a respiratory condition such as asthma, administration of the composition comprising benzaldehyde is by inhalation. According to some such embodiments, benzaldehyde is present in the composition at a concentration of from 0.5% v/v to 5.0% v/v. In some embodiments, the benzaldehyde concentration in the composition is from about 0.08% v/v to about 1.0% v/v, or about 1.0% v/v or about 1.5wtw % or about 2.0% v/v or about 2.5% v/v or about 3.0% v/v or about 3.5% v/v or about 4.0% v/v or about 4.5% v/v. In some embodiments, the total amount of the composition to be inhaled during a specified period of time, such as a day, is up to 600 ml.

According to some embodiments, the composition is administered in a food, such as being mixed into the food.

According to some embodiments, the composition is administered in the drinking water of a non-human animal.

According to some embodiments, the carrier comprises a single carrier. According to some embodiments, the carrier comprises two or more carriers.

According to some embodiments, the carrier is water.

According to some embodiments, the carrier is an organic solvent, such as an alcohol.

According to some embodiments, the carrier comprises water and an organic solvent, such as an alcohol.

According to some embodiments, the composition further comprises at least one excipient. According to some embodiments, the excipient is an emulsifier such as propylene glycol.

According to some such embodiments, the composition comprises vitamin C. The present inventors have surprisingly found that in compositions comprising benzaldehyde and vitamin C, absorption of both the benzaldehyde and of vitamin C is enhanced. According to some embodiments, vitamin C is present in the composition in an amount of between 3 mg and 2 g, or about 0.05% v/v.

According to some embodiments, benzaldehyde is present in the composition at a concentration of from about 0.08% v/v to about 50% v/v, such as about 0.08% v/v, about 0.1% v/v, about 0.2% v/v, about 0.5% v/v, about 1.0% v/v, about 2.0% v/v, about 3.0% v/v, about 4.0% v/v, about 5.0% v/v, about 6.0% v/v, about 7.0% v/v, about 8.0% v/v, about 9.0% v/v, about 10% v/v, about 15% v/v, about 20% v/v, about 25% v/v, about 30% v/v, about 35% v/v, about 40% v/v, about 45% v/v or about 50% v/v.

According to some embodiments, benzaldehyde is present in the composition at a concentration of about 10% v/v.

According to some embodiments, the benzaldehyde is provided at a concentration of from about 0.25% v/v to about 5% v/v, or from about 0.5% v/v to about 3% v/v, or from about 0.8% v/v to about 1% v/v, or from about 1% v/v to about 2% v/v, or from about 1% to about 1.5% v/v in a liquid composition. All ranges described herein are understood to include the end points of the ranges.

According to some embodiments, a dosage of the composition for administration is proportional to a body weight of the subject. For example the dosage for administration may be determined such that between 1.5 to 2.5 ml of the mixture is administered to the subject per 10 kilos body weight. For example a person who weighs 70 kilos would receive 10.5 to 17.5 cc of the mixture. In some embodiments, the benzaldehyde is about 5% by volume of the solution or other mixture. In other embodiments, the benzaldehyde is 3% or is 4% or is 5% or is 6% or is 7% by volume.

According to some embodiments, the composition is for administering to an average human subject having a weight of about 70 kg at a daily dosage of from about 0.5 ml to about 1000 ml, such as about 0.5 ml, about 1.0 ml, about 2.0 ml, about 5.0 ml, about 10 ml, about 20 ml, about 30 ml, about 40 ml, about 50 ml, about 60 ml, about 70 ml, about 80 ml, about 90 ml, about 100 ml, about 200 ml, about 300 ml, about 400 ml, about 500 ml, about 600 ml, about 700 ml, about 800 ml, about 900 ml or about 1000 ml. According to some embodiments, the composition is for administering at a daily dosage of from about 0.5 ml to about 50 ml or from about 0.5 ml to about 30 ml of a composition comprising benzaldehyde at a concentration of from about 0.5% v/v to about 15% v/v, or from 0.8% v/v to about 1.0% v/v of the total composition.

According to some embodiments, the composition is for administering to the subject at least once a day (such as in the morning or in the evening), twice a day (such as once in the morning and once in the evening), three times a day, or more than three times a day at various times of the day. According to some embodiments, the composition is for administering to the subject once a week, twice a week, three times a week, four times a week, five times a week, six times a week, once a month, twice a month or three times a month.

According to some embodiments, the composition is provided in a form selected from the group consisting of a solid, a semi-solid, a liquid and a vapor.

According to some embodiments, the solid form is a powder. According to some such embodiments, the carrier is silica.

According so some embodiments, the solid form is a tablet or capsule.

According to some embodiments, wherein the carrier is water, the composition comprises an emulsion.

According to some embodiments, wherein the carrier comprises an organic solvent, the composition comprises a homogenous solution.

The composition of the present invention may be administered by any route of administration known in the art, including enteral and parenteral routes.

According to some embodiments, administering comprises oral administration.

According to some embodiments, the composition is provided in the form of a vaporizer liquid or a mist generated from a solution of the benzaldehyde in water, and administering comprises inhalation of a vaporized form or mist of the liquid, such as emitted by a vapor-producing device, such as a vaporizer.

In one non-limiting example, the vapor producing device is a vaporizer or humidifier of the type commonly used by parents to deliver medications or medicaments to sleeping children. In other embodiments, the vapor producing device is a device that converts a liquid into a mist or vapor and constantly emits the resulting mist or vapor that has been produced. The amount of time that the patient is situated near the vapor producing device or in the same room as the vapor producing device can vary as long as the person is configured to inhale the vapors. In some embodiments, the vapor is emitted near the person or patient or in the same room as the person or patient for at least three minutes or about 5 minutes or at least five minutes or at least 7 minutes or about 10 minutes at least ten minutes or for 10-15 minutes or at least 15 minutes or longer. According to some such embodiments, the vaporized or mist form is inhaled by the subject for at least five minutes.

According to some embodiments, the semi-solid form is a cream and the method comprises topical administration.

According to some such embodiments, benzaldehyde is present in a cream for topical administration at a concentration of from 0.1% v/v to 3.2% v/v. In certain embodiments, the concentration is about 1.0% v/v or about 1.5% v/v or about 2.0% v/v or about 3.0% v/v. In certain embodiments, the concentration is from about 0.8% v/v to about 1.0%. According to some embodiments, the cream may further comprise one or more other active ingredients, or excipients, such as those needed to maintain the cream in stable form, including, for example, thickeners, emulsifying agents, preservatives, antioxidants, and buffer agents.

According to some embodiments, when the composition is for treatment or prevention of coronavirus infection, such as a COVID19 infection or to reduce the severity of symptoms thereof, the composition may be provided in an ingestible form, in a form suitable for injection, such as intravenous injection, or in vapor form. According to some such embodiments, the benzaldehyde is present in the composition at a concentration of from about 0.08% v/v, such as from 3% v/v to about 15% v/v, from 3% v/v to 7% v/v, or from 5% v/v to 15% v/v.

In some embodiments, the absolute quantity is determined such that between 1.5 to 2.5 ml of the solution or other mixture is administered to the subject for each 10 kilos of a body weight of the subject.

According to some embodiments, the subject is a human subject.

According to some embodiments, the subject is a non-human subject, including a non-human mammal, a bird, a fish, poultry, a farm animal such as cattle or other livestock, a domestic animal and the like. According to some embodiments of the present invention, the composition is for use in a method of treating or preventing a disease in livestock, comprising administering by feeding the livestock with an animal feed or drink comprising benzaldehyde, optionally at a concentration of from about between 0.08% v/v to about 15% v/v, such as between 0.08% v/v and 3.2% v/v, between 5% v/v and 15% v/v, between 3% v/v and 7% v/v, between 0.8% v/v and 1.0% v/v or at about 3%, 4%, 5%, 6% or 7% v/v of the total composition. In some embodiments, the composition is added in the form of a liquid composition or as a spray.

In some embodiments, as a result of the presence of the benzaldehyde, which provides an antimicrobial effect, it is not necessary to add antibiotics to the feed, such that in some embodiments, the feed is devoid of an antibiotic other than the benzaldehyde. The benzaldehyde solution or other mixture is thus a substitute for other antibiotics commonly added to animal feeds.

According to an aspect of some embodiments of the present invention, there is provided a method of controlling growth of a plant, comprising contacting the plant with a composition comprising benzaldehyde and a carrier. According to some embodiments, the method prevents growth of a plant by preventing multiplication of the plant cells. According to some embodiments, the plant is first allowed to grow to a desired size and then contacted with the composition in order to prevent further growth, while maintaining the plant in a healthy state.

According to some embodiments, the composition may be applied to the plant in the form of a spray; sprinkled onto the plant in the form of a powder; or applied in the form of a liqud, such as by applying directly to a surface of the plant or by adding to a water supply which is provided to the plant, such as by any known irrigation system, including manual irrigation, drip irrigation, spray irrigation and the like.

According to some embodiments, benzaldehyde is present in a composition, such as a liquid composition, for controlling growth of a plant at a concentration in the range of from about 0.8% v/v to about 3.2% v/v of the total composition, such as from about 0.8% v/v to about 1.0% v/v, from about 1.0% v/v to about 1.5% v/v, or from about 2.0% v/v to about 3.0% v/v of the total composition.

According to an aspect of some embodiments of the present invention, there is provided a method of preserving food comprising introducing a composition comprising benzaldehyde and a carrier into a vicinity of the food. According to some such embodiments, the benzaldehyde prevents decomposition of the food due to growth of microorganisms or due to undesirable chemical changes. According to some embodiments, the composition for contacting with the food is provided in the form of a solid, liquid or gas. According to some embodiments, the composition is directly contacted with the food. According to some embodiments, the composition is not directly contacted with the food but is provided as a gas in the vicinity of the food. According to some embodiments, the gas is introduced into a container, such as a bag, box, carton or the like containing the food.

According to an aspect of some embodiments of the present invention, there is provided a method of preserving a tissue comprising contacting the tissue with a composition comprising benzaldehyde and a carrier.

According to some embodiments, the composition may be sprinkled on the food or tissue in liquid or powder form. According to some embodiments, the composition may be applied to the food or tissue in vapor form. According to some embodiments, the composition may be provided within a package or container in which the food or tissue is to be stored.

According to some embodiments, the composition for use as a food preservative, comprises benzaldehyde at a concentration of from about 0.25% v/v to about 7% v/v, such as about 0.5% v/v, 1.0% v/v, 1.5% v/v, 1.75% v/v, 3% v/v, 4%/v/v, 5% v/v, 6% v/v or 7% v/v of the total composition. In some embodiments, the composition is provided in the form of a spray for spraying on a food item, such as fish, meat or bread.

According to some embodiments, the composition for use as a food preservative comprises benzaldehyde at a concentration of between 0.25% v/v and 1.25% v/v in water, combined with or applied to a food (such as meat, fish or bread).

According to some embodiments, the composition for use as a food preservative comprising benzaldehyde at a concentration of between 0.25% v/v and 1.25% v/v in water combined with or incorporated into a portion of a food container.

According to an aspect of some embodiments of the present invention, there is provided a method of disinfecting a surface, comprising contacting the surface with a composition comprising benzaldehyde and a carrier.

According to some such embodiments, the surface is selected from the group consisting of an area of skin of an animal and a surface of an inanimate object.

According to some embodiments, the area of skin is an area on the face, hands, feet or torso of an animal.

According to some embodiments, the surface of an inanimate object is a surface of an object such as an item of furniture, a kitchen or bathroom surface, a keyboard or keypad of an electronic device or the like.

According to some embodiments, the composition for use as a disinfectant comprises benzaldehyde at a concentration of from about 0.5% v/v to about 15.0% v/v. In certain embodiments, the concentration of the benzaldehyde in the composition is from about 0.8wtw % to about 1.0% v/v, or about 1.0% v/v, about 1.5% v/v, about 2.0% v/v, about 2.5% v/v, about 3.0% v/v, about 3.5% v/v, about 4.0% v/v or about 4.5% v/v of the total composition.

According to any one of the embodiments disclosed herein, the benzaldehyde is synthetic benzaldehyde.

According to any one of the embodiments disclosed herein, the benzaldhyde is obtained from a part of a plant of the order Rosales, wherein the part of the plant is selected from the group consisting of a pit, a seed and a fruit. According to some such embodiments, the plant of the order Rosales is selected from the group consisting of roses, strawberries, blackberries and raspberries, apples and pears, plums, peaches and apricots, almonds, rowan and hawthorn, jujube, elms, banyans, figs, mulberries, breadfruit, nettles, hops, and cannabis.

According to some such embodiments, the benzaldehyde is obtained by distillation and/or extraction. According to some such embodiments, the part of the plant is contacted with an extractant, wherein benzaldehyde is extracted into the extractant. Additionally or alternatively, the extractant comprising the benzaldehyde may be distilled, such as by steam distillation.

According to an aspect of some embodiments of the present invention, there is provided a nutritional supplement comprising a composition comprising benzaldehyde and a carrier.

According to some such embodiments, the nutritional supplement, such as a vitamin substitute or supplement comprises benzaldehyde at a concentration of from about 0.08% v/v to 15% v/v, such as from about 0.08% to 1.0%) of the composition. According to some embodiments, the nutritional supplement is administered once or twice a day.

According to some embodiments, nutritional supplement is administered at a daily amount of from 0.5 ml to 50 ml, and in one non-limiting example the composition may be taken as a vitamin supplement or replacement once or twice daily. In certain examples, the daily dosage of the supplement is about 0.5 ml, about 1 ml, about 5 ml, about 10 ml, about 20 ml, about 25 ml, about 30 ml, about 35 ml, about 40 ml, about 45 m 1 or about 50 ml.

According to some embodiments, the nutritional supplement is an additive for an animal feed, such as an additive to a livestock feed. According to some such embodiments, the additive comprises benzaldehyde at a concentration of from about 0.8% v/v and 15% v/v, such as from 0.8% v/v to 3.2% v/v, or from about 5% v/v to 15% v/v of the composition. According to some such embodiments, the composition is in the form of a liquid or a spray. In some embodiments, the livestock feed does not include antibiotics.

According to an aspect of some embodiments of the present invention, there is provided a kit for use in treating or preventing an infection in a subject in need thereof, the kit comprising benzaldehyde and a vaporizer, said vaporizer having a receptacle configured to receive therein a composition comprising the benzaldehyde and to vaporize said composition. According to some such embodiments, the receptacle is configured to receive a volume of at least 5 ml of the composition and to emit the vaporized liquid in a predetermined direction. According to some embodiments, benzaldehyde is present in the composition for vaporization at a concentration of from about 0.8% v/v to about 15% v/v, such as from about 0.8% v/v to about 3.2% v/v, from about 3% v/v to about 7% v/v, or from about 5% v/v to about 15% v/v.

According to some embodiments of the kit, the benzaldehyde is provided as a composition in a liquid carrier. According to some embodiments, the kit further comprises separately the benzaldehyde and a liquid carrier for combination of the benzaldehyde with the carrier in the receptacle prior to use.

According to some embodiments, the kit is for use in treating or preventing infection with COVID19 coronavirus in a subject.

According to some embodiments, the kit further comprises instructions to a user as to how to vaporize and inhale the mixture for treating and/or preventing an infectious disease, the instructions appearing on printed material or a computer disc configured to display the instructions on a display

According to an aspect of some embodiments of the present invention, there is provided a food package comprising an inner volume for containing therein a food product and a composition comprising benzaldehyde and a carrier. According to some such embodiments, the composition is separated from the inner volume by a barrier, which may be moved, removed or broken prior to use.

According to some embodiments of the food package, the benzaldehyde is present in the composition at a concentration of from about 0.08% v/v and 15% v/v of the total composition. According to some such embodiments, the benzaldehyde is present in the composition provided to the food package at a concentration of 3% v/v and 7% v/v of the total composition, such about 3% v/v, 4% v/v, 5% v/v, 6% v/v or 7% v/v of the total composition.

### Method of Preparation

According to an exemplary embodiment, benzaldehyde for use in preparation of the composition of the present invention may be obtained from almonds or other portions (for example pits or seeds or petals) of plants that are members of the Rosales order of flowering plants (such as the seeds of apples, pits of apricots or seeds of cherries or pits of peaches etc.).

According to an embodiments, the almonds are soaked for at least 24 hours in an extractant such as water, then the water containing the extract is subjected to steam distillation (at around 80-140 degrees Celsius) to obtain a distillate that contains benzaldehyde, which is then diluted with water to obtain the desired concentration of benzaldehyde.

According to an alternative embodiment of a method of preparation of a composition comprising benzaldehyde, a synthetic version of benzaldehyde is provided, to which is added water and optionally an emulsifier.

According to some embodiments, the benzaldehyde is dried is to provide a powder comprising benzaldehyde and a carrier, such as silica, in powder form. According to some such embodiments, the benzaldehyde is present in the powder composition at a concentration of between about 0.8% v/v and 15% v/v, such as from 0.8% v/v to 3.2% v/v, about 10% v/v, about 5% v/v to about 15% v/v, about 0.5% v/v to 5% v/v, about 0.8% v/v to 1.0% v/v, or about 0.1% v/v to 3.2% v/v.

### Example 1—A Laboratory Study on Fish

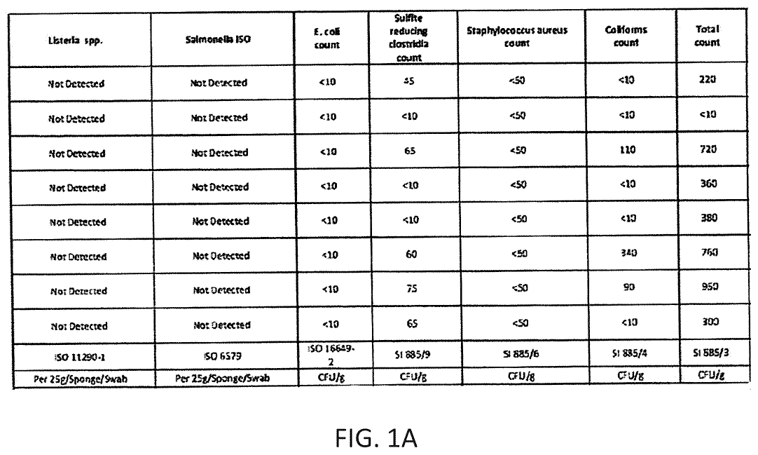

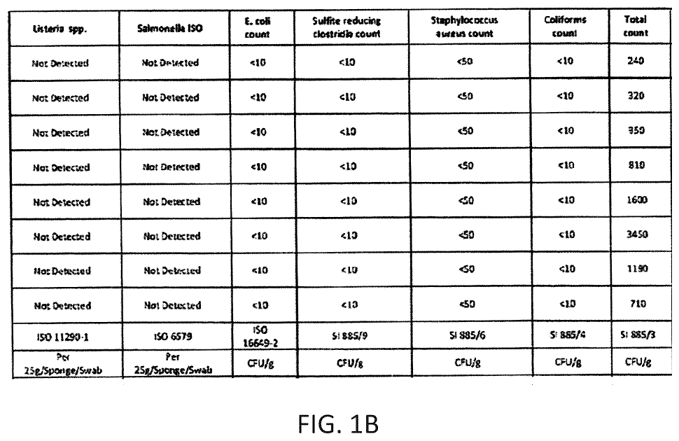

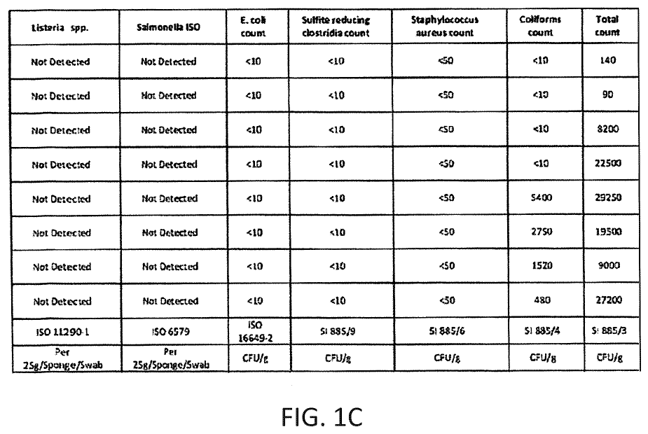

As shown in FIG. 1A, FIG. 1B, FIG. 1C, under laboratory conditions performed by the Institute for Food Microbiology in Israel during August through September 2020, a mixture of benzaldehyde in water (5% by volume) was poured on killed tilapia (the English translation of the fish that in Hebrew is called Amnoon), a freshwater fish that feeds on plant life. The fish were divided into four treated groups labeled “T”, “T1”, “T2” and “T3”. A corresponding amount (by weight) of fish were divided into four control groups and labeled as “S”, “S1”, “S2” and “S3”. “T1”, “T2” and “T3”. The fish of the treated groups were treated with a composition comprising 5% benzaldehyde in ice water.

The amount of specific bacteria was measured for each treated group and control group of fish at the following three times: after 10 minutes (FIG. 1A), after 5 days of continuous treatment (FIG. 1B) and after 12 days of continuous treatment (FIG. 1C). In each case, the total bacterial count was also measured and the results appear in specific columns depending on the specific bacteria (Listeria spp., Salmonella ISO, E. coli, Sulfite reducing clostridia. Staphylococcus aureus, Coliforms and total bacterial count). The results are presented in in FIGS. 1A-1C.

The results show that the treated groups of fish showed marked reduction in bacteria levels. For example, following a period of 12 days from fishing to test and application of the benzaldehyde 5% composition, the following results were obtained: The total count for the treated fish was 140. The total count for the control group was 29,250. For the Coliforms (measured in CFU/G) the results were <10 for the treated group and 5,400 for the control group.

The results showed a very significant reduction in bacteria for fish that were treated with the composition comprising benzaldehyde 5% compared to the control group.

### Example 2—A Study of Food Preservatives

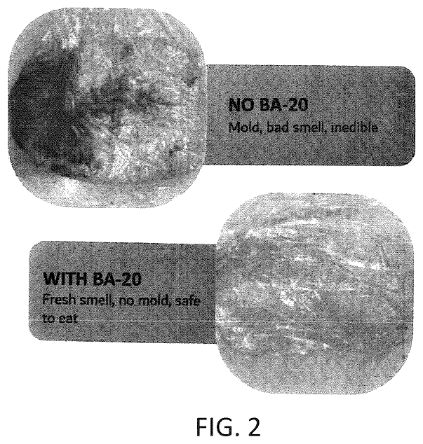

As shown in FIG. 2, under laboratory conditions, a composition comprising benzaldehyde in water (5% by volume) was sprayed on a test group of foodstuffs in water, including fish, meats, and bread in ice water. No treatment was applied to a control group of foodstuffs. The foodstuffs were in ice water and were sprayed with the mixture of benzaldehyde 5% in water showed freshness even after days outside the refrigerator. The same foodstuffs in ice water that were not sprayed with the formaldehyde mixture were moldy, had bad smell and were inedible.

### Example 3—Observational Study

A method in accordance with the principles of the present invention was observationally tested on patients diagnosed as suffering from COVID-19 to evaluate the efficacy of a treatment regimen consisting of a composition comprising benzaldehyde in water with the benzaldehyde being about 5% of the mixture by volume. Each patient drank the mixture twice a day, for example once in the morning and once at night. The absolute amount drunk was based on body weight. For each 10 kilo of body weight the amount drunk was 2 cc. For example, a person who weighed 70 kilo drank 14 cc. The results of the observational study showed that oral administration of the mixture to patients suffering from COVID-19 (including fever, lung congestion, infection) stopped the progression and deterioration of moderately to severely ill patients.

In these patients, significant improvement was noted during the observation period. Specifically, within 48-72 hours, Covid 19 symptoms abated.

The majority of patients were non-hospitalized patients. In a small percentage of the total number of patients, the patients were hospitalized patients but were not undergoing any additional COVID19 treatment because they were told to wait. In a few cases, the patients were undergoing a standard regimen of COVID19 treatment to which they were not responding and that continued during the administration of the benzaldehyde mixture. In these patients, significant improvement was noted during the observation period. Specifically, within 48-72 hours, Covid 19 symptoms abated.

### Experiment to Determine Concentration for Use in Destroying Bacteria

The following is a summary of an experiment using the benzaldehyde composition as disclosed herein to destroy bacteria, for example to test it as a preservative of food. The benzaldehyde was tested for its ability to inhibit the growth of microbes at ten different concentrations of the benzaldehyde, each concentration was half of the previous concentrations as a result of a dilution using TSB as the medium. The following concentrations were tested by successive dilutions of 100% benzaldehyde: 50%, 25%, 12.5%, 6.25%, 3.125%, 1.5625%, 0.78125%, 0.390625%, 0.195313%, 0.097656%. Each concentration appears in the chart below for each of five different strains of bacteria. The experiment was conducted twice and therefore generated two charts (see below). The presence of a “+” in the box indicates that the particular bacteria was found and the presence of a “−” indicates that the particular bacteria was not found. Thus, the minimum concentration needed to substantially eliminate the bacterial strain can be deduced from the experiment.

The initial concentration of the microbes was approximately 103. The microbes studied are listed below. Results are presented in Table 1 below.
1. Enterococcus feaclis
2. Staphylococcus aureus
3. Pseudomonas aeruginosa
4. E. coli
5. Klebsiella pneomoniae

TABLE 1

| Benzaldehyde concetration/ | | | | | | | | | | |
|---|---|---|---|---|---|---|---|---|---|---|
| bactria | 50 | 2 | 12.5 | 6.25 | 3.125 | 1.5625 | 0.78125 | 0.390625 | 0.195313 | 0.097656 |
| enterococcus | − | − | − | + | + | + | + | + | + | + |
| staph. Aureus | − | − | − | − | − | + | + | + | + | + |
| PS | − | − | − | − | − | − | + | + | + | + |
| E. coli | − | − | − | − | − | − | + | + | + | + |
| Kleb. Pneumoniae | − | − | − | − | − | − | + | + | + | + |

A further experiment was conducted in which instead of starting from a 100% concentration, we started from a 10% concentration of benzaldehyde and performed successive dilutions. However, even at the first dilution to a concentration of 5%, there was a positive detection of bacteria for all strains. This experimental was not repeated after receiving the results. Results are presented in Table 2 below.

TABLE 2

| Benzaldehyde concentration/ | | | | | | | | | | |
|---|---|---|---|---|---|---|---|---|---|---|
| bacteria | 50 | 25 | 12.5 | 6.25 | 3.125 | 1.5625 | 0.78125 | 0.390625 | 0.195313 | 0.097656 |
| enterococcus | − | − | − | + | + | + | + | + | + | + |
| staph. Aureus | − | − | − | − | − | − | + | + | + | + |
| PS | − | − | − | − | − | − | + | + | + | + |
| E. coli | − | − | − | − | − | − | + | + | + | + |
| Kleb. Pneumoniae | − | − | − | − | − | − | − | − | + | + |

It was concluded that an antibacterial effect against at least some bacteria is obtained at benzaldehyde concentrations as low as 1.5625%, while a broad spectrum effect is obtained at concentrations of from 12.5%.

### Microbiological Testing of a Composition in Powder Form

A composition comprising 10% v/v benzaldehyde in powder form was prepared to determine the antimicrobial effectiveness of the composition against various microorganisms.

The powder was diluted (2 gram in 2 ml water). Pads saturated with the powder in solution were placed in a closed bag containing the sample to be tested. As negative control, a pad saturated with water alone was placed with the samples in a closed bag.

The microbial suspensions were plated on TSA plates (streaking technique) in duplicates, wherein the microbial suspensions comprised the following microorganisms:

TABLE 3

| ATCC | Strain |
|---|---|
| 14917 ATCC | Lactobacillus Plantarum |
| 27853 ATCC | Pseudomonas aeroginosa |
| 43300 ATCC | Staphylococcus aureus |
| 13048 ATCC | Enterobacter aerogenes |
| 8739 ATCC | E. coli |
| 51299 ATCC | Enterococcus faecalis |
| 19115 ATCC | Listeria monocytogenes |
| 14028 ATCC | Salmonella thyphymoricum |
| 2601 ATCC | Saccharomyces cerevisiae |
| 16404 ATCC | Aspergillus niger |

One plate of each duplicate set was incubated with a pad saturated with the powder in solution and the second plate with water.

The plates were incubated at 30-35° C. for 48 hours and then visually inspected, except for the plates containing A. niger, which were incubated at 20-25° C. and examined after 5 days.

Five different food products were tested: fresh parsley, salmon, pastrami, soft cheese and fresh meat, with and without the saturated pads and negative control. The food samples were tested at time zero and again after one week and two weeks at 7-8° C.

Results are presented in Tables 4 and 5 below:

TABLE 4
microbial isolates

| | Test | Negative |
|---|---|---|
| Bacteria/Yeast/Mould | sample | control |
| E. aerogenes ATCC 13048 | growth | growth |
| P. aeroginosa ATCC 27853 | growth | growth |
| E. coli ATCC 8739 | growth | growth |
| Staphylococcus aureus ATCC 43300 | growth | growth |
| Aspergillus niger ATCC 16404 | Limited | growth |
| | growth | |
| Lactobacillus plantarum ATCC 14917 | growth | growth |
| Enterococcus faecalis ATCC 51299 | growth | growth |
| Listeria monocytogenes ATCC 19115 | growth | growth |
| Salmonella thyphymurium ATCC 14028 | growth | growth |

TABLE 5
food samples

| | | Time one week | | Time two weeks | |
|---|---|---|---|---|---|
| [symbol] | Time zero | Test | NC | Test | NC |
| Parsley | Total count - | Total count - | Total count - | Total count - | Total count - |
| | 300 cfu/gram | 200 cfu/gram | 150 cfu/gram | 1200 cfu/gram | 3000 cfu/gram |
| | Yeasts &moulds - | Yeasts &moulds - | Yeasts &moulds - | Yeasts &moulds - | Yeasts &moulds - |
| | 160/gram | 60/gram | 150/gram | 400/gram | 1200/gram |
| | Coliforms - | Coliforms - <10 | Coliforms - | Coliforms - | Coliforms - |
| | 200 cfu/gram | cfu/gram | 200 cfu/gram | 20 cfu/gram | 800 cfu/gram |
| Salmon | Total count - | Total count - | Total count - | Total count - | Total count - >30,000,000/gr |
| | 700 cfu/gram | 46,000 cfu/gram | 5,800,000 cfu/gram | 340,000 cfu/gram | cfu/gram |
| | Yeasts &moulds - | Yeasts &moulds - | Yeasts &moulds - | Yeasts &moulds - | Yeasts &moulds - >3000/gram |
| | 20/gram | 7 [symbol] 0/gram | 1700/gram | 1200/gram | Coliforms - >3,000,000 |
| | Coliforms - | Coliforms - | Coliforms - | Coliforms - | cfu/gram |
| | 2500 cfu/gram | 36,000 cfu/gram | 1,200,000 cfu/gram | 123,000 cfu/gram | |
| Meat | Total count - | Total count - | Total count - | Total count - | Total count - >3,000,000 |
| | 130,000 cfu/gram | 410,000 cfu/gram | 7,100,000 cfu/gram | 2,300,000 cfu/gram | cfu/gram |
| | Yeasts &moulds - | Yeasts &moulds - | Yeasts &moulds - | Yeasts &moulds - | Yeasts &moulds - |
| | 100/gram | 150/gram | 280/gram | 1000/gram | 1200/gram |
| | Coliforms - | Coliforms - | Coliforms - | Coliforms - | Coliforms - >3,000,000 |
| | 45,000 cfu/gram | 160,000 cfu/gram | 280,000 cfu/gram | 1,300,000 cfu/gram | cfu/gram |
| Pastrami | Total count - | Total count - | Total count - | Total count - >3,000,000 | Total count - >3,000,000 |
| | 45,000 cfu/gram | 210,000 cfu/gram | 350,000 cfu/gram | cfu/gram | cfu/gram |
| | Yeasts &moulds - | Yeasts &moulds - | Yeasts &moulds - | Yeasts &moulds - >300,000/gram | Yeasts &moulds - >300,000/gram |
| | 23,000/gram | 350,000/gram | 280,000/gram | Coliforms - <10 | Coliforms - <10 |
| | Coliforms - <10 | Coliforms <10 | Coliforms - <10 | cfu/gram | cfu/gram |
| | cfu/gram | cfu/gram | cfu/gram | | |
| cheese | Total count - | Total count - | Total count - | Total count - >3,000,000 | Total count - >3,000,000 |
| | 10,000 cfu/gram | 120,000 cfu/gram | 9 [symbol] 0,000 cfu/gram | cfu/gram | cfu/gram |
| | Yeasts &moulds - | Yeasts &moulds - | Yeasts &moulds - | Yeasts &moulds - >300,000/gram | Yeasts &moulds - >300,000/gram |
| | 2300/gram | 100,000/gram | 168,000/gram | Coliforms - | Coliforms - |
| | Coliforms - <10 | | | | |
| | cfu/gram | | | | |

[symbol]

indicates data missing or illegible when filed

According to the test results, the antiseptic composition comprising benzaldehyde in powder form has an antimicrobial activity against molds and certain bacteria. The antimicrobial effect is particularly evident in the study using food products after one week of refrigeration. Significantly reduced microbial counts were observed for salmon and meat after one week when stored with the pad comprising the composition of the present invention.

While the invention has been described with respect to a limited number of embodiments, it will be appreciated that many variations, modifications and other applications of the invention may be made. Therefore, the claimed invention as recited in the claims that follow is not limited to the embodiments described herein.

1. A method of treating or preventing a condition in a subject in need thereof, the method comprising administering to the subject a composition comprising benzaldehyde and a carrier.

2. The method of claim 1, wherein the condition is selected from the group consisting of cancer, heart disease, cataracts, poor eyesight, an immune deficiency, poor brain function, an inflammatory condition, a viral condition, a bacterial condition, hypercholesterolemia, poor iron absorption, a respiratory disease, arteriosclerosis, dementia, tissue damage, skin damage due to exposure to sun, diabetes, age-related degenerative disorders, stroke, cerebrovascular incidents, asthma and shortness of breath, and poor respiratory system.

3. The method of claim 2, wherein said cancer is selected from the group consisting of breast cancer, ovarian cancer, lung cancer, uterine cancer, and lungs, helps prevent uterine cancer, breast cancer, prostate cancer, lung cancer and cancer of the large intestine.

4. The method of claim 2, wherein said viral condition comprises coronavirus infection.

5. The method of any one of claims 1 to 4, wherein the composition is administered in a food.

6. The method of any one of claims 1 to 5, wherein benzaldehyde is present in the composition at a concentration of from about 0.08 wt % to about 50 wt %.

7. The method of claim 6, wherein benzaldehyde is present in the composition at a concentration of about 10 wt %.

8. The method of claim 6 or claim 7, wherein the composition is administered at a daily dosage of from about 0.5 ml to about 1000 ml.

9. The method of any one of claims 1 to 8, wherein the composition is provided in a form selected from the group consisting of a solid, a semi-solid, a liquid and a vapor.

10. The method of claim 9, wherein the solid form comprises a powder.

11. The method of claim 9, wherein the carrier is water and the composition comprises an emulsion.

12. The method of claim 9, wherein the carrier comprises an organic solvent and the composition comprises a homogenous solution.

13. The method of any one of claims 1 to 12, wherein administering comprises oral administration.

14. The method of claim 9, wherein the composition is a liquid and wherein administering comprises inhalation of a vapor produced from said composition.

15. The method of claim 9, wherein the semi-solid form is a cream and the method comprises topical administration.

16. The method of any one of claims 1 to 15, wherein the composition further comprises vitamin C.

17. A method of controlling growth of a plant, comprising contacting the plant with a composition comprising benzaldehyde and a carrier.

18. A method of preserving food comprising introducing a composition comprising benzaldehyde and a carrier into a vicinity of the food.

19. A method of disinfecting a surface, comprising contacting the surface with a composition comprising benzaldehyde and a carrier.

20. The method of claim 19, wherein the surface is selected from the group consisting of an area of skin of an animal and a surface of an inanimate object.

21. The method of any one of claims 1 to 20, wherein the benzaldehyde is synthetic benzaldehyde.

22. The method of any one of claims 1 to 20, wherein the benzaldehyde is isolated from a part of a plant of the order Rosales, wherein the part of the plant is selected from the group consisting of a pit, a seed and a fruit.

23. The method of claim 22, wherein said benzaldehyde is isolated by a method selected from the group consisting of steam distillation and extraction.

24. A nutritional supplement comprising a composition comprising benzaldehyde and a carrier.

25. A kit for use in treating or preventing an infection in a subject in need thereof, the kit comprising benzaldehyde and a vaporizer, said vaporizer having a receptacle configured to receive therein a composition comprising the benzaldehyde and to vaporize said composition.

26. The kit of claim 25, wherein said benzaldehyde is provided as a composition in a liquid carrier.

27. The kit of claim 25, further comprising a liquid carrier for combination with said benzaldehyde in said receptacle.

28. A food package comprising an inner volume for containing therein a food product and a composition comprising benzaldehyde and a carrier.

29. The food package of claim 28, wherein said composition is separated from said inner volume by a removable barrier.
